(19) United States
(12) Patent Application Publication
(10) Pub. No.: US 2024/0355461 A1
(43) Pub. Date: Oct. 24, 2024

(54) HEALTHCARE MANAGEMENT SYSTEM AND METHOD

(71) Applicant: Smile Bridge, LLC, Sheridan, WY (US)

(72) Inventor: Jared Robert Burr, Simpsonville, SC (US)

(73) Assignee: Smile Bridge, LLC, Sheridan, WY (US)

(21) Appl. No.: 18/305,917

(22) Filed: Apr. 24, 2023

(51) Int. Cl. G16H 40/20; G06Q 20/10

(52) U.S. Cl. CPC G16H 40/20; G06Q 20/10

(57) ABSTRACT

A healthcare management system is described. The system may include one or more nodes associated with a blockchain. The one or more nodes may include a blockchain ledger having patient information. The nodes may further include a processor configured to obtain a request from a patient to avail a medical procedure from a medical provider. The processor may obtain the patient information from the blockchain ledger responsive to obtaining the request. The processor may determine that a health saving account associated with the patient is linked with healthcare management system, and schedule an appointment for the medical procedure with the medical provider when the health saving account is linked with the system. In addition, the processor may determine a medical procedure authenticity, and deduct an amount associated with the medical procedure from the health saving account responsive to a determination that the medical procedure is authentic.

### TECHNICAL FIELD

The present disclosure relates to healthcare management system and method, and more particularly, to system and method for facilitating medical treatment, processing payment, and determining medical treatment authenticity using blockchain technology.

### BACKGROUND

Medical insurance companies provide insurance benefits to users. The users may be required to may premium to avail the insurance benefits. The premium may be paid by employer associated with the users or directly by the users.

When a user avails a medical treatment, for example, a dental treatment from a dental practitioner, the dental practitioner (or practitioner's office) may submit a claim to respective insurance company for reimbursement of dental treatment fee. The insurance company may review the claim and pay the dental treatment fee to the dental practitioner. In this arrangement, while the user benefits from not having to pay directly to the dental practitioner, the dental practitioner may be required to coordinate with the insurance company to receive the payment. This may cause inconvenience to the dental practitioner.

In addition, in some instances, the user may schedule an appointment with the dental practitioner but may not show up for the dental treatment at the appointment date and time. This may cause further inconvenience to the dental practitioner, as the dental practitioner may lose appointment timeslot and the insurance company may not pay for the appointment that the user may have missed.

Thus, there exists a need for system and method that facilitate medical treatment and payment for the medical treatment.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts a block diagram of an example node in accordance with the present disclosure.

FIG. 3 depicts a flow diagram of an example first method to facilitate medical treatment in accordance with the present disclosure.

FIG. 4 depicts example snapshots of a user interface to facilitate medical treatment in accordance with the present disclosure.

FIG. 5 depicts a flow diagram of an example second method to facilitate medical treatment, in accordance with the present disclosure.

### DETAILED DESCRIPTION

### Overview

The present disclosure describes healthcare management system and method that may facilitate medical treatment and payment for the medical treatment. The system may include a blockchain ledger that may be configured to store information associated with a patient. For example, the blockchain ledger may include medical records and financial transaction records/information associated with the patient. The blockchain ledger may be a decentralized and distributed information source that may be continuously updated, distributed and/or stored in the system. The system may be configured to obtain a request from the patient to avail a medical procedure from a medical provider. Responsive to obtaining the request, the system may determine whether health saving account (HSA) associated with the patient is linked with the healthcare management system based on the financial information stored in the blockchain leger. Responsive to a determination that the HSA is linked, the system may enable the patient to schedule an appointment with the medical provider. The system may be further configured to process payment associated with the medical procedure from the patient's HSA to an account associated with the medical provider when the medical procedure is performed. The system may be further configured to store information associated with the medical procedure in the blockchain ledger (e.g., payment information in the financial information and medical procedure in the medical records). In some aspects, responsive to a determination that the patient's HSA is not linked with the healthcare management system, the system may prompt the patient to link the HSA with the system before enabling the patient to schedule the appointment.

The system may be further configured to determine if the patient has missed the appointment. Responsive to a determination that the patient may have missed the appointment, the system may process a payment of a predefined amount from the patient's HSA to the account associated with the medical provider. The system may be further configured to store information associated with the missed appointment and corresponding predefined amount in the blockchain ledger.

In further aspects, the system may be configured to determine medical procedure authenticity when the medical provider performs the medical procedure on the patient. In this case, the system may estimate or determine medical inventory that may be used in the medical procedure that the patient may desire to undergo and compare the estimated inventory with actual medical inventory that may be removed from a medical warehouse to determine the medical procedure authenticity. For example, the system may receive inputs from Radio-Frequency Identification (RFID) tags that may be disposed on the medical inventory that may be located in the medical warehouse to track actual medical inventory that the medical provider may use during appointment date and time. The system may determine the medical procedure to be authentic if the estimated medical inventory matches with at least one actual medical inventory that may be removed from the medical warehouse during the appointment date and time. In some aspects, the system may process the payment associated with the medical procedure when the system determines that the medical procedure may be authentic. The system may be further configured to store information associated with the medical procedure authenticity in the blockchain ledger (e.g., in the medical records).

In additional aspects, the system may determine the medical procedure authenticity based on sterilization status of medical inventory that the medical provider may use in the medical procedure. In this case, the system may receive inputs from the RFID tags indicating whether the medical/surgical tools used in the medical procedure are (or were) sterilized during the appointment date and time. The system may determine the medical procedure to be authentic when the inputs from the RFID tags indicate that the medical/surgical tools used in the medical procedure during the appointment date and time were sterilized. The system may be further configured to store information associated with medical inventory sterilization status in the blockchain ledger.

The present disclosure discloses system and method that may facilitate medical treatment and payment for the medical treatment. The system enables the medical provider to receive medical treatment fee or amount directly from the patient HSA when the medical procedure is performed. In addition, the system enables the medical provider to receive a predefined amount (or fee) when the patient misses the appointment. In addition, the system processes the payment after determining medical procedure authenticity that prevents fraudulent medical activity and ensures that all the tools used in the medical procedure are sterilized.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

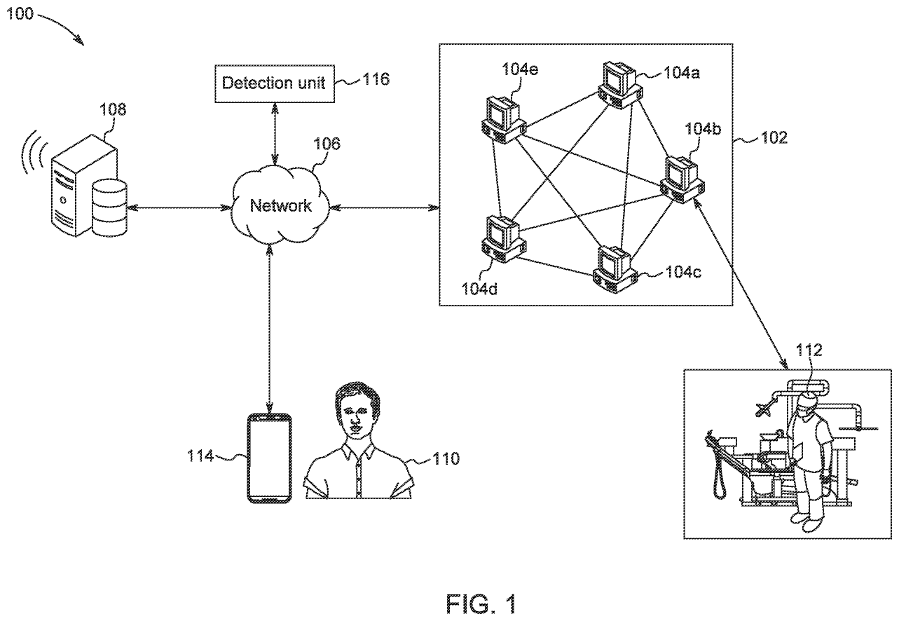

FIG. 1 depicts an example environment 100 in which techniques and structures for providing the systems and methods disclosed herein may be implemented. The environment 100 may include a healthcare management system 102 or a distributed healthcare network 102. The healthcare management system 102 may be a blockchain system and may include a plurality of nodes 104a, 104b, 104c, 104d, 104e (collectively referred as nodes 104) that may be connected with each other via a communications network 106 (or network 106). Hereinafter, the healthcare management system 102 is referred to as the distributed healthcare network 102.

The nodes 104 may represent nodes in a healthcare blockchain system. A person ordinarily skilled in the art may appreciate that blockchain represents a technology that may decentralize data control between nodes (such as the nodes 104) on a network (such as the network 106), generate and examine immutable records on a public ledger, and exchange assets between the nodes 104. Blockchain may refer to a growing list of records, called “blocks”, that may be linked to one another using cryptographic techniques.

In some aspects, the nodes 104 may be associated with different entities including, but not limited to, doctors/medical resources, hospitals, etc. For example, the node 104a may be associated with a hospital, the node 104b may be associated with a doctor such as a dental practitioner 112 or a medical provider 112, the node 104c may be associated with another doctor (e.g., another dental practitioner or physician), and/or the like. The nodes 104 may include mobile phones, laptops, computers, servers, tablets, and/or other similar devices having communication capabilities associated with the entities described above.

The network 106 may be, for example, a communication infrastructure in which the connected devices discussed in various embodiments of this disclosure may communicate. The network 106 may be and/or include the Internet, a private network, public network or other configuration that operates using any one or more known communication protocols such as, for example, transmission control protocol/Internet protocol (TCP/IP), Bluetooth®, BLE®, Wi-Fi based on the Institute of Electrical and Electronics Engineers (IEEE) standard 802.11, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few examples.

The environment 100 may further include a server 108 that may be an external server (i.e., may not be part of the distributed healthcare network 102) that may be connected with the nodes 104 via the network 106. In some aspects, the server 108 may be associated with health saving account (HSA) associated with a plurality of subscribers (e.g., a patient 110). Specifically, the server 108 may store information associated with health saving accounts of the plurality of subscribers. For example, the server 108 may store subscriber names, contact details, total contributions made by the subscribers in the HSA, current balance in the HSA, and/or the like. The subscribers may use the HSA to pay for medical expenses defined by an authority. For example, the subscribers may use the HSA to pay for dental treatment.

In some aspects, each node 104 may be configured to perform similar functionality. For example, the node 104 may be a computing platform and may be configured to provide medical assistance in scheduling appointments, making medical treatment payment, etc. to the patient 110 via a patient user device 114. In some aspects, the patient user device 114 may be part of the distributed healthcare network 102. In other aspects, the patient user device 114 may be outside (i.e., may not be part of) the distributed healthcare network 102 (as depicted in FIG. 1).

In an exemplary aspect, the patient 110 may desire to avail a medical service/procedure from the medical provider 112 (such as the dental practitioner). The patient 110 may transmit, using the patient user device 114, a request for the medical service/procedure to the node 104 (for example, to the node 104b associated with the medical provider 112). The node 104 may receive the request and determine whether an HSA associated with the patient 110 is linked with the distributed healthcare network 102. Responsive to determining that the HSA associated with the patient 110 may be linked with the distributed healthcare network 102, the node 104 may enable the patient 110 to schedule an appointment with the medical provider 112 to avail the medical procedure. The node 104 may further facilitate payment processing (associated with the medical procedure availed by the patient 110 from the medical provider 112) from the patient's HSA to a medical provider account when the patient 110 avails the medical procedure.

In additional aspects, the node 104 may be configured to determine that the patient 110 may have missed a scheduled appointment with the medical provider 112 for the medical procedure. Responsive to determining that the patient 110 may have missed the appointment, the node 104 may facilitate payment of a predefined amount or fee for the missed appointment from the patient's HSA to the medical provider account.

In further aspects, the node 104 may be connected with a detection unit 116 via the network 106. The detection unit 116 may be configured to detect or track medical inventory or artifacts that may be used by the medical provider 112 in the medical procedure/service that the patient 110 may avail. In some aspects, the detection unit 116 may be based on Radio-Frequency Identification (RFID) technology. Specifically, the detection unit 116 may include RFID tags disposed on the medical inventory to track medical inventory usage (e.g., when the medical inventory may be loaded in a medical warehouse, when the medical inventory may be removed from the medical warehouse for the medical procedure, etc.). The node 104 may be configured to receive inputs from the detection unit 116 and may be configured to determine authenticity of the medical procedure performed by the medical provider 112 on the patient 110. In some aspects, the node 104 may be configured to process the payment associated with the medical procedure responsive to a determination that the medical procedure may be authentic.

In further aspects, the detection unit 116 may be configured to track sterilization of medical/surgical tools that may be used for the medical procedure. Stated another way, the medical/surgical tools may include RFID tags that may enable the node 104 to determine whether the medical/surgical tools that may have been used for the medical procedure on the patient 110 are sterilized. The node 104 may process the payment associated with the medical procedure responsive to a determination that the medical/surgical tools may be sterilized.

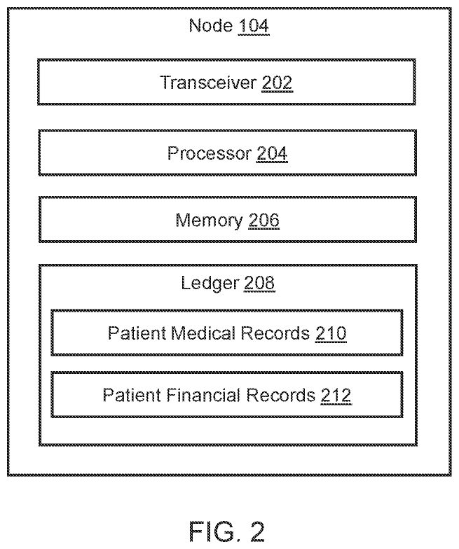

In addition, the node 104 may be configured to facilitate completion of formalities between the patient 110 and the medical provider 112. For example, the node 104 may be configured to obtain a consent form from a node memory (shown as memory 206 in FIG. 2) when the patient 110 visits the medical provider 112 for a medical procedure (e.g., root canal). Responsive to obtaining the consent form, the node 104 may enable the patient 110 to read and sign the consent form (on the node 104) before the medical provider 112 starts the medical procedure. The node 104 may obtain the signed consent form and store the form in the node memory. The details of the node 104 may be understood in conjunction with FIG. 2.

FIG. 2 depicts a block diagram of an example node (e.g., the node 104b associated with the medical provider 112) in accordance with the present disclosure. Each node 104 (including the node 104b) may include a transceiver 202, a processor 204, a memory 206 and a ledger 208 (or blockchain ledger 208). The transceiver 202 may be configured to transmit/receive instructions or information to/from other nodes in the distributed healthcare network 102. In addition, the transceiver 202 may be further configured to receive information or data from a device located outside distributed healthcare network 102. For example, the transceiver 202 may be configured to receive request from the patient 110 to schedule appointment with the medical provider 112, patient's HSA information from the server 108, medical inventory/tools tracking information associated with medical inventory/tools used in the medical procedure from the detection unit 116, and/or the like. In addition, the transceiver 202 may be configured to transmit instructions or information to a device located outside distributed healthcare network 102. For example, the transceiver 202 may be configured to transmit request to the detection unit 116 to provide medical inventory/tool information, notification to the patient user device 114 to indicate the medical procedure authenticity to the patient 110 when the medical procedure is performed, and/or the like.

In some aspects, the memory 206 may store programs in code and/or store data for performing various operations in accordance with the present disclosure. Specifically, the processor 204 may be configured and/or programmed to execute computer-executable instructions stored in the memory 206 for performing various functions in accordance with the disclosure. Consequently, the memory 206 may be used for storing code and/or data code and/or data for performing operations in accordance with the present disclosure.

In one or more aspects, the processor 204 may be disposed in communication with one or more memory devices (e.g., the memory 206 and/or one or more external databases (not shown in FIG. 1)). The memory 206 can include any one or a combination of volatile memory elements (e.g., dynamic random-access memory (DRAM), synchronous dynamic random-access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.).

The memory 206 may be one example of a non-transitory computer-readable medium and may be used to store programs in code and/or to store data for performing various operations in accordance with the disclosure. The instructions in the memory 206 can include one or more separate programs, each of which can include an ordered listing of computer-executable instructions for implementing logical functions.

The ledger 208 may be a decentralized and distributed information source that may be continuously updated, distributed and/or stored at the node 104. Other nodes (such as nodes 104a, 104c, 104d, and 104e) in the distributed healthcare network 102 may have access to the ledger 208. The other nodes may have a similar ledger 208 at any given time. The ledger 208 may include or store information associated with the patient 110 including, but not limited to, patient medical records 210 and patient financial records/information 212. The patient medical records 210 may include medical inventory tracking information associated with medical inventory used in the medical procedure (obtained from the detection unit 116), a medical procedure type that may be availed by the patient, date and time of the medical procedure, and historical medical records associated with different services or medical procedures availed by the patient via different medical providers/resources. The patient financial records 212 may include the patient 110 HSA details (e.g., account number, HSA balance etc.), payment information associated with the medical procedures/services availed by the patient 110, (e.g., payment made from the patient's HSA to different medical providers corresponding to different medical services availed by the patient), and/or the like. In addition, the ledger 208 may include patient personal information (such as name contact details), patient unique identifier etc. In some aspects, the ledger 208 may obtain the above-described information from an external device/server (e.g., the server 108) when the ledger 208 does not already have some or all information. Further, in some aspects, the ledger 208 (and the nodes 104) may be Health Insurance Portability and Accountability Act (HIPPA) compliant.

The process of facilitating the patient 110 to avail the medical services from the medical provider 112 by using the node 104 is described in detail below in conjunction with subsequent figures.

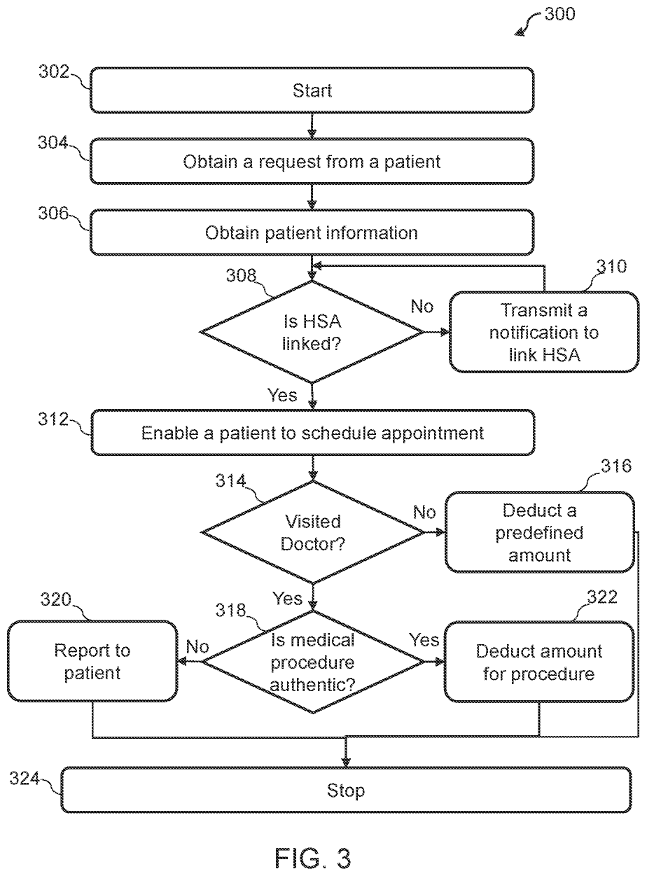

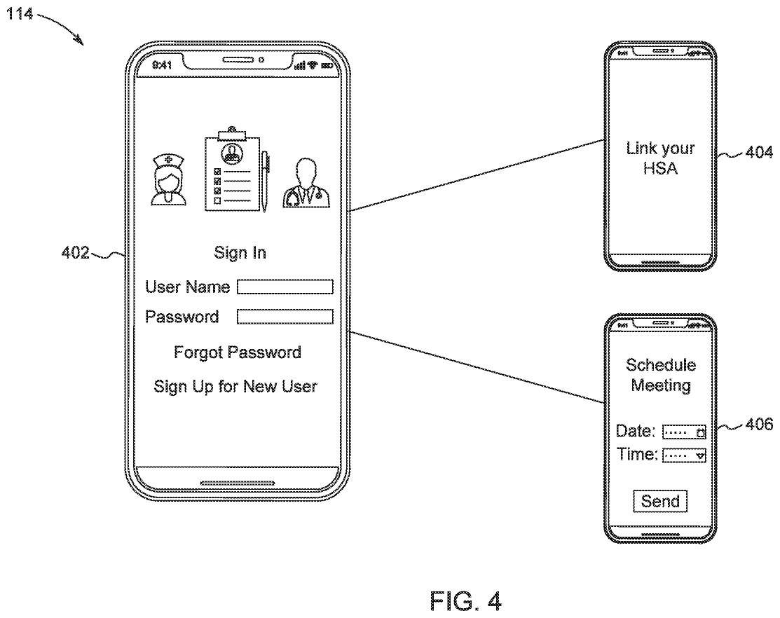

FIG. 3 is a flowchart of an example first method 300 to facilitate medical treatment in accordance with the present disclosure. FIG. 3 may be described with continued reference to FIGS. 1 and 2. The following process is exemplary and not confined to the steps described hereafter. Moreover, alternative embodiments may include more or less steps that are shown or described herein and may include these steps in a different order than the order described in the following example embodiments. While describing FIG. 3, references will be made to FIG. 4. Specifically, FIG. 4 depicts example snapshots of a user interface to facilitate the medical treatment in accordance with the present disclosure.

Referring to FIG. 3, at step 302, the method 300 may commence. At step 304, the method 300 may include obtaining, by the processor 204, a request from a patient user device (for example, the patient user device 114 described in conjunction with FIG. 1) associated with the patient 110 via the network 106. Specifically, the transceiver 202 may receive a request from the patient 110 (via the patient user device 114) to avail the medical procedure from the medical provider 112. The request may indicate the medical procedure that the patient 110 may desire to undergo. In some aspects, the patient 110 may log-in to a portal associated with the distributed healthcare network 102 by using the patient user device 114 (e.g., the patient 110 may enter patient name, password, and/or other login credentials to log-in to the portal, as shown in view 402 of FIG. 4), and may transmit the request to the transceiver 202 to avail the medical procedure from the medical provider 112 via the portal.

Responsive to receiving the request from the patient user device 114, the transceiver 202 may transmit the request to the processor 204. The processor 204 may obtain the request from the transceiver 202, for example, when the transceiver 202 receives the request from the patient user device 114.

At step 306, the method 200 may include obtaining, by the processor 204, patient information from the ledger 208, responsive to obtaining the request. For example, the processor 204 may obtain the patient financial records/information 212 associated with the patient 110 from the ledger 208. At step 308, the method 300 may include determining, by the processor 204, whether the HSA associated with the patient 110 is linked with the distributed healthcare network 102.

Responsive to a determination that the HSA may not be linked, the method 300 may move to step 310. At step 310, the method 300 may include transmitting, by the processor 204 via the transceiver 202, a notification/request to the patient user device 114 instructing the patient 110 to link the HSA with the distributed healthcare network 102, as shown in view 404 of FIG. 4. Responsive to receiving the notification/request on the patient user device 114, the patient 110 may input details of the HSA on the portal to link the HSA (e.g., name, password, and/or other information) with the distributed healthcare network 102. In a scenario where the HSA is not setup for the patient 110, the patient 110 may sign up for the HSA, and may add an amount in the HSA. For example, the patient 110 may transmit, via the patient user device 114, a request to the server 108 to setup the HSA via the network 106. Alternatively, the patient 110 may transmit, via the patient user device 114, the request to setup the HSA to the server 108 via the node 104 and the network 106. Responsive to receiving the request, the server 108 may ask for patient's personal information and create the HSA responsive to receiving patient's personal information.

On the other hand, responsive to a determination that the HSA may be linked with the distributed healthcare network 102 at step 308, the method 300 may move to step 312. At step 312, the method 300 may include enabling, by the processor 204, the patient 110 to schedule an appointment for the medical service/procedure from the medical provider 112, as shown in view 406 of FIG. 4. For example, when the patient 110 transmits the request to avail dental treatment from a dental practitioner, the processor 204 may confirm if the HSA associated with the patient 110 is linked with the distributed healthcare network 102. Responsive to determining that the HSA may be linked with the distributed healthcare network 102, the processor 204 may enable the patient 110 to schedule an appointment with the dental practitioner for the dental treatment.

In some aspects, to enable the patient 110 to schedule the appointment, the processor 204 may obtain medical provider availability from a calendar associated with the medical provider 112 (e.g., date and time at which the patient 110 may visit the dental practitioner). The processor 204 may then display the medical provider availability (e.g., different days and corresponding time slots) to the patient 110 on the patient user device 114. The patient 110 may view the medical provider availability on the patient user device 114, select a date/time on the patient user device 114 and provide a confirmation to the transceiver 202 to schedule the appointment at the selected date/time. The processor 204 may obtain the confirmation from the transceiver 202 and may schedule the appointment for the patient 110 with the medical provider 112. In some aspects, the processor 204 may store information associated with the appointment in the ledger 208 (e.g., in the patient medical records 210).

In additional aspects, to enable the patient 110 to schedule the appointment, the processor 204 may cause the patient user device 114 to display a list of services (and corresponding cost and/or medical procedure or service code) provided by the medical provider 112. For example, the processor 204 may transmit, via the transceiver 202, the list of services to the patient user device 114 and the patient user device 114 may display the list of services. The patient 110 may view the list on the patient user device 114 and may select one or more services from the list of displayed services for the medical procedure. The service may include regular dental diagnosis or specific dental treatment. In some aspects, the patient 110 may select the service after a discussion with the medical provider 112. The patient 110 may select the service at the time of scheduling the appointment.

In addition, responsive to a determination that the HSA may be linked with the distributed healthcare network 102 and when the patient 110 schedules the appointment, the processor 204 may obtain/fetch medical records associated with the patient 110 from the ledger 208 (e.g., using the unique identifier associated with the patient 110). Specifically, the processor 204 may obtain historical medical records including time and date information with respect to different services availed by the patient 110 historically through different medical providers/resources. In some aspects, the processor 204 may display the obtained medical records on a user interface associated with the node 104 or a user device associated with the medical provider 112 before (or when) the patient 110 visits the medical provider 112 (e.g., during the appointment) for the medical procedure.

At step 314, the method 300 may include determining, by the processor 204, whether the patient 110 has visited the medical provider 112 at scheduled appointment date/time. Responsive to a determination that the patient 110 may not have visited the medical provider 112 at the scheduled appointment date/time or missed the appointment, the method 300 may move to step 316. At step 316, the method 300 may include deducting, by the processor 204, a predefined amount/fee from the HSA associated with the patient 110 for the missed appointment. In additional aspects, the processor 204 may select a procedure code associated with missed appointment and may store the selected code in the patient medical records 210 associated with the patient 110 (in the ledger 208). In further aspects, the processor 204 may store information associated with the predefined amount/fee in the ledger 208 (e.g., in the patient financial records 212). The method 300 then moves to step 324.

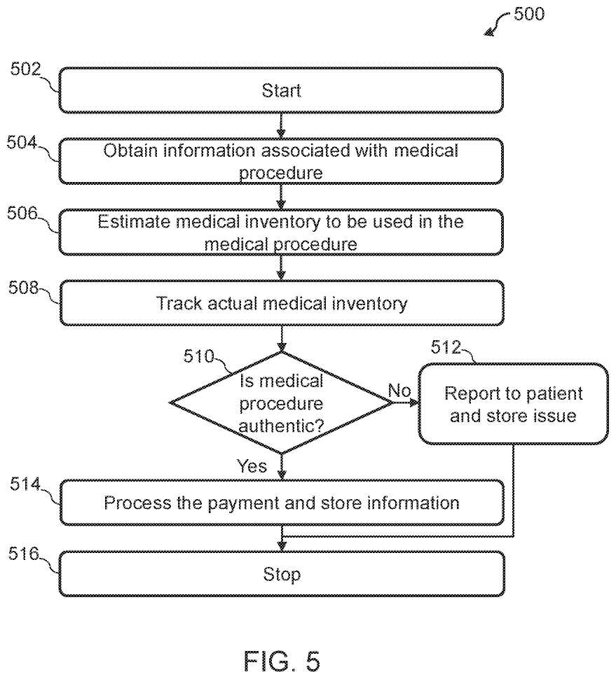

On the other hand, responsive to a determination that the patient may have arrived for the appointment, the method 300 moves to step 318. At step 318, the method 300 may include determining, by the processor 204, medical procedure authenticity associated with the medical procedure availed by the patient 110. In some aspects, the processor 204 may store medical procedure information associated with the medical procedure in the ledger 208 (e.g., in the patient medical records 210) when the medical procedure is performed (e.g., when the scheduled appointment may be over). The medical procedure information may include, but not limited to, medical inventory tracking information associated with medical inventory/tools used in the medical procedure (obtained from the detection unit 116), a medical procedure type (e.g., root canal treatment, dental implant etc.) that may be availed by the patient 110, and medical procedure date and time, etc. The processor 204 may determine the medical procedure authenticity based on the medical procedure information. The details of determining medical procedure authenticity may be understood in conjunction with FIG. 5.

Responsive to a determination that the medical procedure may not be authentic at step 318, the method 300 may move to step 320. At step 320, the method 300 may include reporting, by the processor 204, issue to the patient 110. For example, the processor 204 may transmit, via a transceiver 202, a notification to the patient user device 114. The notification may indicate that the medical procedure may not be authentic. In addition, the processor 204 may be further configured to store such information associated with the authentication in the ledger 208. For example, the processor 204 may store such issue in the patient medical records 210 associated with the patient 110. In some aspects, the processor 204 may not initiate payment process from the patient's HSA to an account associated with the medical provider 112 when the processor 204 determines that the medical procedure may not be authentic. The method 300 then moves to step 324.

On the other hand, responsive to a determination that the medical procedure may be authentic at step 318, the method 300 may move to step 322. At step 322, the method 300 may include deducting, by the processor 204, an amount associated with the medical procedure/service availed by the patient 110 from the HSA associated with the patient 110. Specifically, the processor 204, at the end of the appointment, may prompt the medical provider 112 to provide a confirmation of the planned medical procedures/services provided during the appointment. Responsive to receiving the confirmation from the medical provider 112, the processor 204 may determine respective amount/fee for each of the respective medical services that may be provided, calculate a total amount for the medical procedure(s), and deduct the total amount from the patient's HSA to be transferred to the medical provider account. In further aspects, the processor 204 may store information associated with the amount in the ledger 208, for example, in the patient financial records 212.

In some aspects, each medical procedure may be associated with a code (e.g., dental procedure code such as Current Dental Terminology (CDT) or International Classification of Disease (ICD)). The processor 204 may be configured to fetch code(s) associated with the medical procedure(s) the patient 110 may have undergone or availed. The processor 204 may be further configured to correlate the codes with medical provider 112 fee for the medical procedure (which may be pre-stored in the memory 206). The processor 204 may be further configured to store the correlation in the ledger 208, for example, in the patient financial records 212. The method 300 then moves to step 324 at which the method 300 stops.

In further aspects, the processor 204 may be configured to use Artificial Intelligence (AI) and/or machine learning to fetch appropriate code for the medical procedure and perform the correlation. A person ordinarily skilled in the art may appreciate that machine learning is an application of the AI using which processor 204 may have the ability to automatically learn and improve from experience without being explicitly programmed. Machine learning focuses on use of data and algorithms to imitate the way humans learn. In some aspects, the machine learning algorithms may be created to make classifications and/or predictions.

Machine learning may be of various types based on data or signals available to the learning system. For example, the machine learning approach may include supervised learning, unsupervised learning, semi-supervised learning, or reinforcement learning. The supervised learning is an approach that may be supervised by a human. In this approach, the machine learning algorithm may use labeled training data and defined variables. In the case of supervised learning, both the input and the output of the algorithm may be specified/defined, and the algorithms may be trained to classify data and/or predict outcomes accurately.

Broadly, the supervised learning may be of two types, “regression” and “classification”. In classification learning, the learning algorithm may help in dividing the dataset into classes based on different parameters. In this case, a computer program may be trained on the training dataset and based on the training, the computer program may categorize input data into different classes. Some known methods used in classification learning include Logistic Regression, K-Nearest Neighbors, Support Vector Machines (SVM), Kernel SVM, Naïve Bayes, Decision Tree Classification, and Random Forest Classification.

In regression learning, the learning algorithm may predict output value that may be of continuous nature or real value. Some known methods used in regression learning include Simple Linear Regression, Multiple Linear Regression, Polynomial Regression, Support Vector Regression, Decision Tree Regression, and Random Forest Regression.

The unsupervised learning is an approach that involves algorithms that may be trained on unlabeled data. An unsupervised learning algorithm may analyze the data by its own and find patterns in input data. Further, semi-supervised learning is a combination of supervised learning and unsupervised learning. A semi-supervised learning algorithm involves labeled training data; however, the semi-supervised learning algorithm may still find patterns in the input data. Reinforcement learning is a multi-step or dynamic process. This model is similar to supervised learning but may not be trained using sample data. This model may learn “as it goes” by using trial and error. A sequence of successful outcomes may be reinforced to develop the best recommendation or policy for a given problem in reinforcement learning.

In an exemplary aspect, the processor 204 may use supervised machine learning technique to identify appropriate code for the medical procedure availed (or to be availed) by the patient 110, and correlate the codes with the medical provider 112 fee. Specifically, the processor 204 may obtain a training/label data that includes codes for different medical procedures and correlation of the codes and medical provider 112 fee. For example, the training data may include fee for regular dental diagnosis and associated code, fee to perform root canal treatment and associated code, and/or the like. The processor 204 may generate a fee module based on the training data. The fee module may be configured to receive new requests (including medical procedure availed or to be availed by the patient 110), identify codes associated with the request, and correlate the codes with the medical provider 112 fee (for new requests) to enable the processor 204 to calculate or estimate total cost for the medical procedure(s).

The method 300 may include one or more additional steps (not shown). For example, in some aspects, the method 300 may include determining, by the processor 204, whether the amount in the patient's HSA may be sufficient to pay the medical provider 112 fee for the medical procedure/services availed by the patient 110. Responsive to determining that the patient's HSA may have sufficient funds to pay for the availed medical procedure/services, the processor 204 may deduct the medical provider 112 fee from the patient's HSA. Alternatively, responsive to determining that the patient's HSA may not have sufficient funds to pay for the availed medical procedure/services, the processor 204 may output a notification to a user interface of the node 104b (or a user device associated with the medical provider 112) indicating the balance/deficit amount that the patient 110 needs to pay to the medical provider 112. In some aspects, the processor 204 may calculate the deficit amount by subtracting the amount/fee associated with the medical procedure and the amount in the HSA.

FIG. 5 depicts a flow diagram of an example second method 500 to facilitate medical treatment in accordance with the present disclosure. FIG. 5 may be described with continued reference to FIGS. 1-4. The following process is exemplary and not confined to the steps described hereafter. Moreover, alternative embodiments may include more or less steps that are shown or described herein and may include these steps in a different order than the order described in the following example embodiments.

Referring to FIG. 5, at step 502, the method 500 may commence. At step 504, the method 500 may include obtaining, by the processor 204, information associated with medical procedure. Specifically, the processor 204 may obtain information associated with the medical procedure that the patient 110 may have undergone, when the patient 110 undergoes the medical procedure. As described above, the patient 110 may indicate in the request (described in step 304 of FIG. 3) the medical procedure that the patient 110 may desire to undergo when the patient 110 schedules the appointment with the medical provider 112. The processor 204 may fetch the information associated with the medical procedure from the request. For example, while scheduling the appointment, the patient 110 may indicate that the patient 110 may desire to schedule the appointment for dental implant procedure. The processor 204 may fetch such information associated with the medical procedure from the request. In addition, the processor 204 may fetch appointment date and time from the request.

At step 506, the method 500 may include estimating, by the processor 204, medical inventory that may be used in the medical procedure based on the obtained information associated with the medical procedure. For example, when the processor 204 obtains information that the medical procedure may be dental implant procedure, the processor 204 may estimate or determine that medical inventory such as dental implant kit, crown, etc. may be required to complete the medical procedure. In some aspects, the ledger 208 or the memory 206 may store information associated with the estimated medical inventory for respective medical procedures. For example, the ledger 208 or the memory 206 may store medical inventory/items required to perform root canal, medical inventory/items (dental kit, medical/surgical equipment, etc.) required to perform dental implant, and/or the like. The processor 204 may estimate the medical inventory that may be used in the medical procedure by fetching information from the memory 206 or the ledger 208 and correlating the fetched information with the obtained information associated with the medical procedure.

At step 508, the method 500 may include tracking, by the processor 204, actual medical inventory used from medical warehouse during appointment date and time (or before the appointment time such as 15 minutes before the appointment time). Specifically, the processor 204 may be configured to track the actual medical inventory by receiving/obtaining inputs from a detection unit (same as the detection unit 116 described in conjunction with FIG. 1). The detection unit 116 may include RFID tags that may be disposed on the medical inventory, and may be configured to detect or track the actual medical inventory. Specifically, the transceiver 202 may obtain medical inventory tracking information associated with the actual medical inventory/items used during the appointment date and time from the detection unit 116, and transmit the information to the processor 204 and the ledger 208 for storage purpose.

At step 510, the method 500 may include determining, by the processor 204, whether the medical procedure may be authentic. Stated another way, the processor 204 may determine whether the medical provider 112 has used estimated medical inventory during the medical procedure based on the medical inventory tracking information, or whether the estimated medical inventory matches with at least one of the actual medical inventories. The processor 204 may determine that the medical procedure may be authentic when the estimated medical inventory matches with the at least one of the actual medical inventories. On the other hand, the processor 204 may determine that the medical procedure may not be authentic when the estimated medical inventory does not match with any one of the actual medical inventories.

For example, the processor 204 may estimate that the dental kit may be required in the medical procedure. The processor 204 may track, via the RFID tags, whether any dental kit was removed during the appointment date and time from the medical warehouse. The processor 204 may determine that the medical procedure may be authentic when the dental kit was taken out from the medical warehouse during the appointment date and time. On the other hand, the processor 204 may determine that the medical procedure may not be authentic when no dental kit was removed from the medical warehouse during the appointment date and time.

In further aspects, the processor 204 may be configured to determine that the medical procedure may be authentic when the medical/surgical tools used in the medical procedure may be sterilized. Specifically, the processor 204 may estimate medical items/equipment to be used in the medical procedure (based on the request, similar to estimation of the medical inventory). The processor 204 may track, via the RFID tags, actual the medical/surgical tools that may be sterilized during the appointment time (or before the appointment time such as 15 minutes before). The processor 204 may then determine whether the estimated medical/surgical tools match with the actual medical/surgical tools that were sterilized during the appointment time and determine the medical procedure authenticity based on the determination. Responsive to a determination that the estimated medical/surgical tools do not match with the actual medical/surgical tools, the processor 204 may determine that the estimated medical/surgical tools may not be sterilized, and thus the medical procedure may not be authentic. On the other hand, when the estimated medical/surgical tools match with the actual medical/surgical tools, the processor 204 may determine that the estimated medical/surgical tools may be sterilized, and thus the medical procedure may be authentic. In further aspects, the processor 204 may store information associated with medical items/equipment sterilization status in the ledger 208 (e.g., in the patient medical records 210).

In some aspects, the processor 204 may perform this step of determining that the medical procedure may be authentic when the appointment time may be over.

In some aspects, the transceiver 202 may store the information associated with actual medical inventory/items used in the medical procedure in the ledger 208, for example, in the patient medical records 210.

Responsive to a determination that the medical procedure may not be authentic, the method 500 may move to step 512. At step 512, the method 500 may include reporting, by the processor 204, issue to the patient 110, as discussed in conjunction with step 320 of FIG. 3. In addition, the processor 204 may be configured to store such information/issue associated with the authentication in the ledger 208. For example, the processor 204 may store such issue in the patient medical records 210 associated with the patient 110. The method 500 then moves to step 516.

Alternatively, responsive to a determination that the medical procedure may be authentic, the method 500 may move to step 514. At step 514, the method 500 may include processing, by the processor 204, payment associated with the medical procedure from the patient's HSA to the account associated with the medical provider 112. In some aspects, the processor 204 may calculate a total cost of the one or more medical procedures that the patient 110 may have undergone during the scheduled appointment. The processor 204 may transmit a notification associated with the total cost to the medical provider 112 and may request the medical provider 112 to provide confirmation on the total cost. The processor 204 may be further configured to receive or obtain confirmation from the medical provider 112 and may initiate the payment associated with the medical procedure(s) from the patient's HSA. In further aspects, the processor 204 may determine if the amount available in the patient's HSA is sufficient to pay for the medical procedure. The processor 204 may further determine a deficit amount (difference between the total cost for the medical procedure and the amount available in the patient's HSA), and transmit a notification to the medical provider 112 to get balance/deficit amount directly from the patient 110 when the amount available in the patient's HSA may be less than the medical procedure fee/cost.

In addition, the processor 204 may store information associated with the payment deducted from the patient's HSA in the ledger 208. For example, the processor 204 may store such information in the patient financial records 212 so that the patient 110 may track the total amount spent on medical expenses/services. The method 500 then moves to step 516.

At step 516, the method 500 may stop.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A healthcare management system comprising:
one or more nodes associated with a blockchain, wherein the one or more nodes comprises:
a blockchain ledger comprising patient information, wherein the patient information comprises patient account information; and
a processor communicatively coupled to the blockchain ledger, wherein the processor is configured to:
obtain a request from a patient to avail a medical procedure from a medical provider;
obtain the patient information from the blockchain ledger responsive to obtaining the request;
determine that a health saving account associated with the patient is linked with the healthcare management system based on the patient information;
enable the patient to schedule an appointment for the medical procedure with the medical provider responsive to a determination that the health saving account is linked with the healthcare management system;
store medical procedure information associated with the medical procedure in the blockchain ledger when the medical procedure is performed;
determine a medical procedure authenticity based on the medical procedure information; and
deduct an amount associated with the medical procedure from the health saving account responsive to a determination that the medical procedure is authentic.

2. The healthcare management system of claim 1, wherein the one or more nodes further comprises a transceiver configured to receive medical inventory tracking information from Radio-Frequency Identification (RFID) tags, wherein the medical inventory tracking information is associated with medical inventory used during the appointment, and wherein the RFID tags are configured to track the medical inventory.

3. The healthcare management system of claim 2, wherein the medical procedure information comprises the medical inventory tracking information, a medical procedure type and medical procedure date and time.

4. The healthcare management system of claim 3, wherein the processor is configured to:
estimate medical inventory to be used in the medical procedure;
determine that the estimated medical inventory is used in the medical procedure based on the medical inventory tracking information; and
determine the medical procedure authenticity based on the determination.

5. The healthcare management system of claim 4, wherein the processor is further configured to store information associated with the medical procedure authenticity in the blockchain ledger.

6. The healthcare management system of claim 4, wherein the processor is further configured to:
estimate medical tools to be used in the medical procedure;
determine that the estimated medical tool is sterilized based on the medical inventory tracking information; and
determine the medical procedure authenticity based on the determination.

7. The healthcare management system of claim 1, wherein the processor is further configured to transmit a request to a patient device requesting the patient to link the health saving account with the healthcare management system responsive to a determination that the health saving account is not linked.

8. The healthcare management system of claim 1, wherein the processor is further configured to determine the amount associated with the medical procedure when the medical procedure is performed.

9. The healthcare management system of claim 8, wherein the processor is further configured to store the amount in the blockchain ledger.

10. The healthcare management system of claim 1, wherein the processor is further configured to:
determine that the patient has missed the appointment; and
deduct a predefined amount from the health saving account responsive to a determination that the patient has missed the appointment.

11. The healthcare management system of claim 10, wherein the processor is further configured to store the predefined amount in the blockchain ledger.

12. The healthcare management system of claim 1, wherein the processor is further configured to:
determine that the amount associated with the medical procedure is greater than an amount available in the health saving account;
calculate a deficit amount by subtracting the amount and the amount available in the health saving account; and
transmit a notification to a medical provider device, wherein the notification comprises the deficit amount.

13. A method to manage a healthcare system, the method comprising:
obtaining, by a processor, a request from a patient to avail a medical procedure from a medical provider;
obtaining, by the processor, patient information from a blockchain ledger responsive to obtaining the request, wherein the blockchain ledger comprises the patient information, and wherein the patient information comprises patient account information;
determining, by the processor, that a health saving account associated with the patient is linked with the healthcare system based on the patient information;
enabling, by the processor, the patient to schedule an appointment for the medical procedure with the medical provider responsive to a determination that the health saving account is linked with the healthcare system;
storing, by the processor, medical procedure information associated with the medical procedure in the blockchain ledger when the medical procedure is performed;
determining, by the processor, a medical procedure authenticity based on the medical procedure information; and
deducting, by the processor, an amount associated with the medical procedure from the health saving account responsive to a determination that the medical procedure is authentic.

14. The method of claim 13 further comprising:
transmitting a request to a patient device requesting the patient to link the health saving account with the healthcare system responsive to a determination that the health saving account is not linked.

15. The method of claim 13 further comprising:
obtaining medical inventory tracking information from Radio-Frequency Identification (RFID) tags, wherein the medical inventory tracking information is associated with medical inventory used in the medical procedure, and wherein the RFID tags are configured to track the medical inventory.

16. The method of claim 15, wherein the medical procedure information comprises the medical inventory tracking information, a medical procedure type, and date and time of the medical procedure.

17. The method of claim 16 further comprising:
correlating appointment timing with the medical inventory tracking information; and
determining the medical procedure authenticity based on the correlation.

18. The method of claim 17 further comprising storing information associated with the medical procedure authenticity in the blockchain ledger.

19. The method of claim 13 further comprising:
determining that the patient has missed the appointment; and
deducting a predefined amount from the health saving account responsive to determining that the patient has missed the appointment.

20. A non-transitory computer-readable storage medium in a distributed computing system, the non-transitory computer-readable storage medium having instructions stored thereupon which, when executed by a processor, cause the processor to:
obtain a request from a patient to avail a medical procedure from a medical provider;
obtain patient information from a blockchain ledger responsive to obtaining the request, wherein the blockchain ledger comprises the patient information, and wherein the patient information comprises patient account information;
determine that a health saving account associated with the patient is linked with a healthcare system based on the patient information;
enable the patient to schedule an appointment for the medical procedure with the medical provider responsive to a determination that the health saving account is linked with the healthcare system;
store medical procedure information associated with the medical procedure in the blockchain ledger when the medical procedure is performed;
determine a medical procedure authenticity based on the medical procedure information; and
deduct an amount associated with the medical procedure from the health saving account responsive to a determination that the medical procedure is authentic.
